(19) United States
(12) Patent Application Publication
(10) Pub. No.: US 2007/0004980 A1
(43) Pub. Date: Jan. 4, 2007

(54) Distributed medical imaging system

(76) Inventors: Adrian Warner, Bothell, WA (US); Kevin Bradley, Bothell, WA (US); Helen Routh, New York, NY (US); Earl Canfield, Snohomish, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(21) Appl. No.: 10/543,793

(22) PCT Filed: Nov. 10, 2003
(86) PCT No.: PCT/IB03/05049

§ 371 Date: May 30, 2006

Related U.S. Application Data
(60) Provisional application No. 60/432,066, filed on Dec. 9, 2002.

(51) Int. Cl. A61B 5/05

(52) U.S. Cl. 600/411

(57) ABSTRACT

A distributed diagnostic imaging system (62) and method includes a data processor coupled to a variety of diagnostic imaging components through a network (80). The diagnostic imaging components include acquisition devices (90) that are used to obtain diagnostic imaging signals, displays (98) on which obtained images can be viewed, and control units (94) that are used with either acquisition units (90) or displays (98) to control the manner in which an image is obtained or displayed. The distributed nature of the system (62) makes it relatively easy and inexpensive to upgrade or modify individual imaging components, and allows the businesses of selling, distributing, and upgrading an imaging lab, and obtaining and reviewing diagnostic images to be conducted in a novel manner, such as by costing an imaging procedure on a “per use” basis.

This invention relates to medical imaging, and more particularly, to medical imaging system architectures that allow the system to be easily configured for specific applications and to be easily upgradeable.

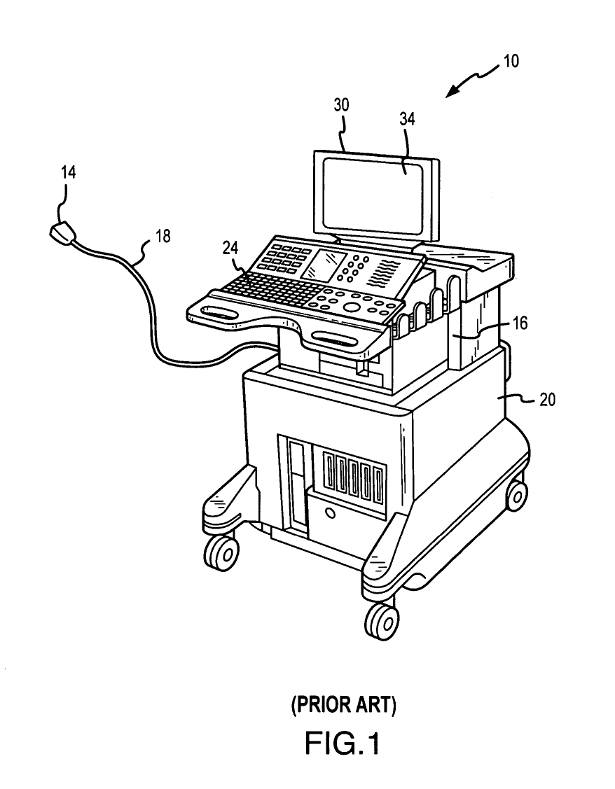

Medical imaging systems, such as diagnostic ultrasound imaging systems, are commonly used to image a wide variety of organs and tissues within the human body. A typical ultrasound imaging system 10 is shown in FIG. 1. The imaging system 10 includes an ultrasound scanhead 14 that is adapted to be placed in contact with a portion of a body that is to be imaged. The scanhead 14 is coupled to a system chassis 16 by a cable 18. The system chassis 16, which is mounted on a cart 20, includes a keyboard and other controls 24 by which data may be entered into a processor (not shown) that is included in the system chassis 16. A display, which may be a cathode ray tube (“CRT”) display or a flat panel display 30 having a viewing screen 34, is located on an upper surface of the system chassis 16.

Ultrasound imaging systems 10 of the type shown in FIG. 1 are called upon to perform a wide variety of tasks in a wide variety of circumstances. For example, in abdominal imaging applications, the quality of the ultrasound images is of paramount importance, and the frame rate, i.e., the rate at which new images can be generated, is of relatively lesser importance. However, in cardiac imaging, the frame rate is of paramount importance to allow the movement of the heart to be accurately visualized in real time or captured in freeze frame. The imaging system 10 should ideally be configurable so that its capabilities can be optimized for each of the functions that it is called upon to perform. It should be possible to select a high frame rate that is desired for cardiac imaging, and yet be able to configure the system to provide the highly resolved images that are desired for abdominal imaging, and so on. In practice, the capabilities of the imaging system 10 are normally limited by economic and technical compromises. In some cases, it may not be technically possible to simultaneously provide all of the capabilities needed for optimum performance of all tasks the imaging system 10 is called upon to perform. For example, the system 10 may be able to provide very high resolution images needed for abdominal imaging, but it may be incapable of doing so at the frame rate needed for cardiac imaging. As a result, the imaging system 10 may be designed to provide images that are less than optimal for abdominal imaging at a rate that is less than optimal for cardiac imaging. Even if it was possible to simultaneously satisfy all technical criteria, the cost of doing so might make the cost of the imaging system 10 prohibitive.

In addition to the performance compromises discussed above, the ultrasound imaging system 10 is also subject to compromises resulting from the manner in which it is used. For example, ultrasound images in the obstetrics field are normally obtained by the patient visiting a location where the machine is located in a hospital or other health care facility. Therefore, for obstetrical imaging, the imaging system 10 need not be compact or portable. However, in other fields or uses, such as when used in an emergency room or an operating room, the imaging system 10 must be moved to the patient since the patient cannot be easily moved. For this reason, the imaging system 10 must be somewhat portable, which is facilitated by making the system compact. Yet it is generally more expensive to make electronic systems more compact. Therefore, the imaging system 10, when used for obstetrics, generally need not be compact, but is preferably more compact and hence more expensive when used for surgery and other fields where the patient comes to the system.

The integrated nature of the ultrasound imaging system 10 is also a factor in the time required to upgrade the performance of the system 10 and implement new features in the system 10. For example, if the capabilities of the keyboard and controls in the system 10 are improved, it is difficult to upgrade only the keyboard and controls since the keyboard and controls are integrated into the remainder of the system 10. Instead, the improved keyboard and controls must generally be implemented in a new imaging system offering.

The above-described problems with and limitations of the stand-alone ultrasound imaging system 10 of FIG. 1 also exists to a greater or lesser degree with medical imaging systems of the other diagnostic imaging modalities, including X-ray, digital radiography, mammography, and computed tomography (“CT”) imaging systems, radiograph systems, magnetic resonance imaging (“MRI”) systems, and PET and nuclear camera systems.

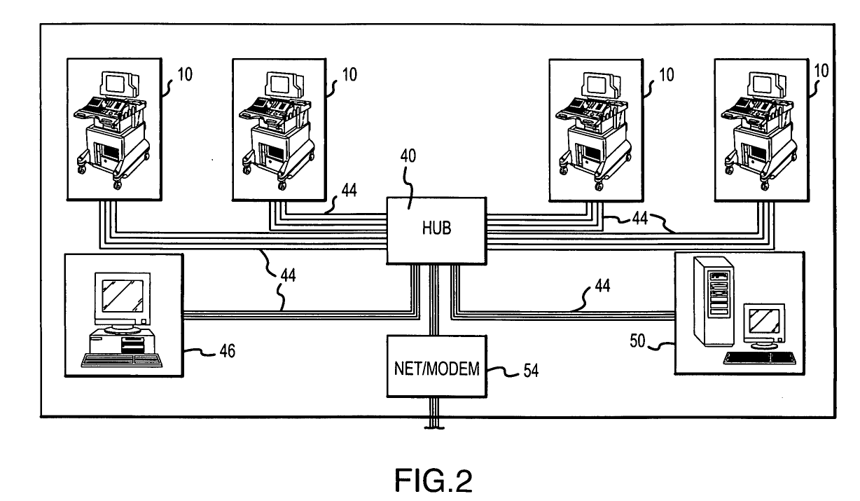

Although imaging systems of the type shown in FIG. 1 are primarily used as a stand-alone unit, they have been used in a network to allow ultrasound images to be communicated to locations away from the system 10. For example, FIG. 2 shows several of the ultrasound imaging systems 10 coupled to a hub 40 though network conductors 44 in a conventional manner. The systems 10 are used to acquire ultrasound images at various locations. The hub 40 is also connected to a personal computer 46, which can be used to examine ultrasound images obtained using the system 10, and a centralized server 50, which can store ultrasound images and make them available for subsequent review and diagnosis. A network coupler or modem 54 is also connected to the hub 40 to allow ultrasound images that are either obtained using the systems 10 or stored by the server 50 to be transmitted elsewhere for remote review and diagnosis.

Another problem with the imaging system shown in FIG. 1 is that it can be difficult to keep track of the ultrasound images obtained and/or viewed using the system 10. If the systems 10 are used as stand-alone systems, there is no way to record usage of the system other than manually. Even if the systems 10 are networked as shown in FIG. 2, there is no established means for tracking the time a system is used for an examination or the number of images obtained or viewed for each patient with whom the system 10 is used. At least for these reasons, it is not feasible to adapt the system 10 to automatically track and charge for use of the system 10 for billing purposes.

While interconnecting ultrasound imaging systems 10 as shown in FIG. 2 allows images generated by the system to be remotely reviewed, it does not eliminate or reduce the problems discussed above. To be economically feasible, the imaging system 10 must still be designed so that its capabilities are a compromise of what is needed to perform each of the functions it will be called upon to perform. Further, although the systems 10 are designed to be compact and portable, those properties are largely wasted by the fact that they are coupled to a network and thus immobile, although using a wireless network can obviate this limitation to some degree. Moreover, when it is necessary or desirable to upgrade the systems 10 which are connected to the network, it is still necessary to install the new hardware or software on all of the systems 10.

There is therefore a need for an ultrasound imaging system in which individual components can be specially adapted to optimally perform a wide variety of functions, and which can be individually upgraded, thereby minimizing the time and expense required to perform such upgrades.

A medical imaging system and method in accordance with the present invention uses a variety of individual imaging components that are coupled through a network to a central system, which performs most of the processing and data storage functions of the system. As a result, each of the individual imaging components, such as acquisition units, displays, and controls, can be optimized to perform each of a variety of specific functions. For example, different acquisition units can be designed for abdominal, cardiac, obstetrical, orthopedic, etc., examinations as well as for different imaging modalities. The entire imaging system can therefore easily and inexpensively be adapted for specific applications simply by using the acquisition device designed for that application or modality. Furthermore, many improvements or upgrades can be made to the system simply by improving or upgrading a single imaging component or a central system, rather than upgrading a multitude of separate imaging machines. Finally, the distributed nature of the imaging system allows charges for purchase or use of the system to be easily made on the basis of such usage. For example, charges can be made for each patient from whom images are obtained, for each image obtained using the system, for each image that is viewed using the system, or for other events reflecting the time or amount of usage of all or part of the system. Furthermore, distributed imaging system are offered to customers as imaging networks rather than self-contained imaging machines, as is the case presently.

In the drawings:

Figure I is an isometric view of a conventional, stand-alone ultrasound imaging system.

FIG. 2 is a block diagram of several ultrasound imaging systems of the type shown in FIG. 1 coupled to each other in a conventional network arrangement.

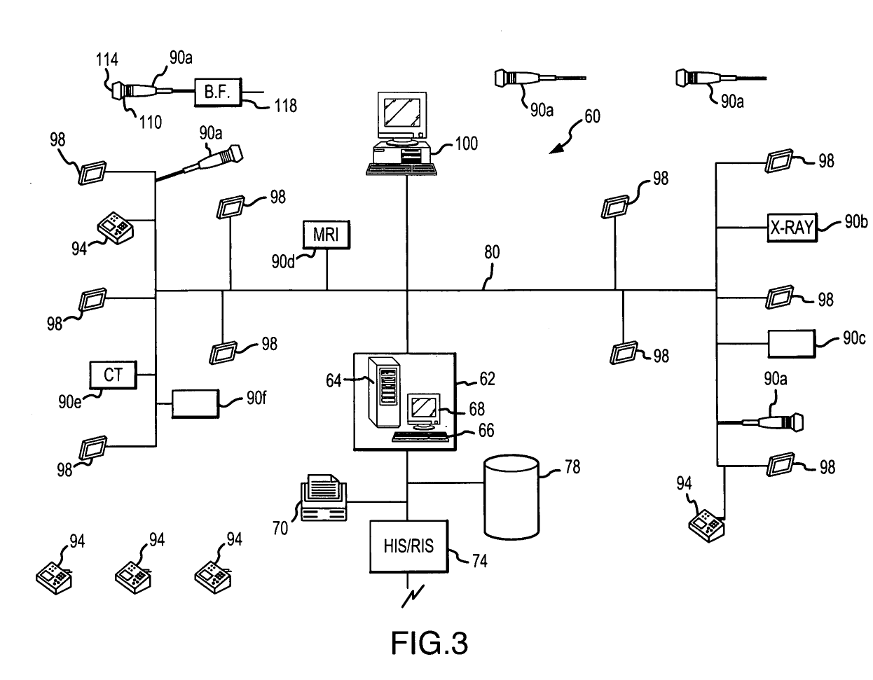

FIG. 3 is a block diagram of a distributed medial imaging system and method according to one embodiment of the invention.

FIG. 3 shows a distributed diagnostic imaging network 60 and method according to one embodiment of the invention. Although the primary function of the network 60 shown in FIG. 3 and described below is to obtain, store and display ultrasound images, it also includes components that allow it to obtain, store and display other types of diagnostic images. The network 60 includes a data processing system 62 that includes a chassis 64, a keyboard 66 and a display monitor 68. Inside the chassis 64 or coupled thereto may be a printer 70, a hospital information system or radiology information system (“HIS/RIS”) 74 and a data storage device 78, such as a disk drive, cineloop, or image archive. The system 62 can be distributed among several processors or servers or p.c.'s, or can comprise the processor of one or more fully integrated imaging systems connected to provide processing capability for a distributed imaging system. As explained below, the system 62 in the illustrated embodiment serves as the central processing unit of the imaging network 60.

The system 62 is coupled to a data network 80, which may be a local area network such as an Ethernet network. Although the network 80 is shown as being a hard-wired network, it will be understood that all or some of the network may be a wireless network, such as a network using the IEEE 802.11 (“WiFi”) protocol, an optical network, or some other type of network. The network 80 may also be coupled to a remote terminal (not shown) through a modem or other device (not shown). For example, the network can be extended to the home of a patient by hard-wired or wireless links to the location where image acquisition occurs.

Coupled to the data network 80 at various locations are a variety of medical imaging components, including acquisition units 90, control units 94, display units 98, and an image review station 100. The locations in the network 80 that these medical imaging components may be connected will depend upon user preference, but can be expected to be in patients' rooms, nurse stations, physicians' or sonographers' offices, radiology and cardiology labs, etc. Additional acquisition units 90, control units 94, and display units 98 are available, preferably at a central storage location, for coupling to the network 80, as shown at the top and bottom of FIG. 3. As shown in FIG. 3, the acquisition units 90 include ultrasound acquisition units 90a, an X-ray acquisition unit 90b, a digital radiography acquisition unit 90c, an MRI acquisition unit 90d, a CT acquisition unit 90e, and a nuclear camera detector 90f. However, it will be understood that not all of these types of acquisition units 90a-f may be coupled to the data network 80, and that image acquisition units 90 other than those shown in FIG. 3 may be coupled to the network 80. Also, all or some of the acquisition units 90a-f, as well as other types of acquisition units 90, may not be coupled to the network at all times but instead may be coupled to the network 90 as needed.

As shown at the upper left-hand corner of FIG. 3, each of the ultrasound acquisition units 90a may include a scanhead 110 formed by one or more transducer elements 114 and, in the case of array transducers, a beamformer 118 that combines signals received from respective transducer elements 114 into a single signal corresponding of ultrasound echoes from body tissues, structures or fluids at multiple angles and depths beneath the ultrasound acquisition unit 90a. The inclusion of a beamformer 118 in the array probes is presently preferred because of the very high bandwidth that would be required in the network 80 if all of the beamforming were performed by the system 62. The use of beamforming circuitry in an acquisition probe is shown, for instance, in U.S. Pat. No. 5,229,933 (Larson III), U.S. Pat. No. 6,142,946 (Hwang et al.), and in U.S. Pat. No. 5,997,479 (Savord et al.) However, with advances in computer and network technology, it may be possible in the future to include only the transducer elements 114 in the ultrasound acquisition units 90a, with the beamforming performed in the system 62.

Each of the ultrasound acquisition units 90a preferably is optimized to obtain a particular type or types of images. For example, each of the ultrasound acquisition units 90a may have a single transducer element 114, a linear array of transducer elements 114 or a two-dimensional array of transducer elements 114. The units 90a may be configured to process signals from the transducer elements 114 to provide two-dimensional images in various planes, such as B-mode images, or they may be configured to provide three-dimensional images. Ultrasound beams from the acquisition units 90a may also be directed in various directions by incorporating mechanical steering devices in the units 90a. The ultrasound acquisition units 90a may also be configured to provide Doppler images in either two or three dimensions. Conventional imaging techniques, such as spatial compounding and harmonic imaging, may also be performed by the units 90a, either alone or under control of the system 62. Furthermore, the operating frequency of the ultrasound acquisition units 90a may also vary as desired. For example, an ultrasound acquisition unit 90a having a relatively high operating frequency, such as 7 MHz, may be used for scanning at relatively shallow depths, but with good resolution. Conversely, an ultrasound acquisition unit 90a having a relatively low operating frequency, such as 3.5 MHz, may be used for scanning at greater depths, although the resolution of the resulting image may be relatively low. Finally, the surfaces of the transducer elements 114 in the ultrasound acquisition units 90a that are placed in contact with patients may be either flat or curved, and, when curved, the units 90a may be curved in a manner that is specifically optimized to obtain an image on a specific part of the body.

In general, a user of the system 10 will normally have available ultrasound acquisition units 90a having various combinations of the parameters discussed above, with each combination being optimized for a particular type of ultrasound examination. When a sonographer or other health care professional is scheduled to conduct a particular type of examination, he or she can simply select the appropriate ultrasound acquisition unit 90a from a storage location, plug the acquisition unit 90a into the network 80, and perform the examination. The examination can be performed at a central location with the patient coming to the sonographer, or the sonographer may go to the patient if, as would be expected, connections to or communicate with the network 80 are readily available at the location of the patient. The other acquisition units 90b-f, as well as image acquisition units not shown in FIG. 3, are used in similar manners.

The control units 94 may also vary depending upon the type of diagnostic image that will be obtained. For obtaining ultrasound images using the network 60, the type of control unit 94 may vary depending on the type of ultrasound examination that will be performed and/or the skill or preference of the sonographer or other heath care professional that will be using the network 60. The control units 94 may, of course, simply replicate many of the control units found on conventional ultrasound imaging units, such as the system 10 shown in FIG. 1. Control units 94 for use with the acquisition units 90b-f for obtaining other types of diagnostic images will vary depending upon the imaging modality and the nature of the image obtained. However, to allow a common control unit 94 to be used with different types of acquisition units 90, the control unit 94 may use “soft keys,” the function of which varies depending upon the type of diagnostic image being obtained. Also, the display units 98 may be provided with “touch screens” or other user interface devices that allows the control of the acquisition units 90 to vary depending on which acquisition unit 90 is being used. In such case, a separate control unit 94 may not be required. Finally, in some cases, the control unit 94 may be integrated into the acquisition units 90, thus making a stand-alone acquisition unit 94 unnecessary.

Although different types of display units 98 can be used, the display units will generally fall into two classes, namely display units 98 that can merely display an image, and display units 98 that are provided with some control functionality, such as the ability to control the brightness or contrast of a displayed image or the parameters used to acquire a displayed image. The display units 98 may have a conventional aspect ratio of 4:3, but they may also have higher aspect ratios, such as a 16:9 aspect ratio, to provide the advantages described in U.S. patent application Ser. No. 09/717,907 to Roundhill, which is incorporated herein by reference. The display units 98 may be implemented using any conventional or hereinafter developed display, such as cathode ray tubes (“CRT”), liquid crystal display (“LCD”) displays, organic light emitting diode (“OLED”) displays, plasma displays, etc. As mentioned above, the display units 98 may also be provided with touch screens or other user interface devices for controlling the acquisition units 90 as well as the display properties of the image presented by the display units 98.

The tasks performed by the system 62 will depend at least in part upon the functionality of the other components of the network 60. Based on presently available technology, the system 62 will perform most of the processing in the network 60. However, with advances in computer and networking technology, it may be possible to incorporate a greater share of the processing power in the acquisition units 90. Alternatively, as previously mentioned, it may also be possible for the system 62 to perform even more of the processing functionality of the system so that the ultrasound acquisition units 90a include only the transducer elements 114. However, in the network 60 shown in FIG. 3, the system 62 couples signals to the ultrasound acquisition units 90a that control the transmitting of ultrasound signals from and the receiving of ultrasound echoes by the ultrasound acquisition units 90a. For example, the signals coupled to acquisition units 90a by the system 62 may trigger a transmission as well as control the frequency and duration of ultrasound signals coupled from the units 90a. The signals coupled to the ultrasound acquisition unit 90a by the system 62 may also control the angle and/or depth from which ultrasound echoes are received. In cases where different ultrasound acquisition units 90a or other ultrasound components in the network 60 have different operating parameters, the operating parameters can be stored either in the component, or may be downloaded to the component from the system 62. Other parameters that are controlled by signals coupled from the system 62 to the ultrasound acquisition units 90a will be apparent to one skilled in the art. The system 62 may also couple signals to either the ultrasound acquisition units 90a or the other acquisition units 90b-f or the display units 98 to set up the acquisition units 90a-f or display units 98 based on the type of image that is to be obtained. Where the system 62 also serves as or is in communication with a hospital information system (“HIS”), the system 62 can automatically configure the acquisition units 90a-f, the control units 94, and/or the display units 98 based on the identity of the patient and the type of examination that is to be performed.

The system 62 may perform a variety of signal processing functions. For example, when an ultrasound image is being obtained, the system 62 may perform some or all of the beamforming in the system, although, as previously indicated, it is presently preferred that most of the beamforming be performed in the ultrasound acquisition units 90a. The system 62 may also perform other signal processing such as harmonic separation, Doppler processing, filtering, demodulation, frequency compounding, or amplitude or quadrature detection on the signals received from the ultrasound acquisition units 90a. The system 62 may also perform various image processing tasks, including scan conversion, spatial compounding, image graphics generation, overlay generation (such as by overlaying a color Doppler image on a B mode image), persistence adjustment, image analysis (such as by detecting an image border), and other graphics processing tasks that will be apparent to one skilled in the art. The processed image is then communicated over the network 80 for display on a display unit 98 used by the clinician operating the acquisition probe which acquired the image information.

The system 62 may also include a report generator module to format and create reports of various types. The nature of such reports will be apparent to one skilled in the art. Also, the system 62 may generate financial documents, such as invoices, to charge for use of the network 60.

The partitioning of software between the system 62 and the acquisition units may be dictated by whether the network is used for a single imaging modality or multiple modalities. For example, the different signal processing functions of the different modalities such as filtering, FFT processing, and Fourier transform processing may remain with the different acquisition units, with only the image processing of the different modalities being performed on the system 62. Upgrades to the software of the acquisition units may still be done by installing the new software on the system 62, then uploading it to the different acquisition units as it is needed or required, and control software for the acquisition units may be resident on the system 62 and uploaded to the acquisition units as needed. As another alternative, some of the image processing unique to the different modalities may remain with the acquisition unit, with only common image processing performed by the system 62. For example, it may be decided to perform the polar to rectilinear scan conversion of ultrasound image data on the ultrasound acquisition units and the back projection reconstruction of CT on the CT acquisition units, while image processing such as DICOM formatting or 3D image rendering applicable to ultrasound, CT, and MRI, for instance, is performed by the system 62.

In operation, the distributed diagnostic imaging network 60 allows a great deal of flexibility in the manner in which the network 60 is operated. For example, a health care professional can optimize the system to obtain a particular type of diagnostic image or to obtain a diagnostic image from a particular part of the body simply by choosing an acquisition unit 90 that is optimized for such purpose. Once diagnostic images have been obtained, they can be examined on individual display units 98 that can merely display an image or display units 98 that are provided with some control functionality, such as the ability to control the brightness or contrast of a displayed image, or a touchscreen that enables the selection of imaging parameters. An acquired diagnostic image can also be reviewed using the image review station 100 or a remote terminal (not shown) through a modem or other communication device coupled to the network 80. Basically, since all of the data corresponding to obtained images are stored by the system 62, such as on data storage unit 78, the images can be examined on any device that can be coupled to the system 62 through the network 80. Furthermore, the data corresponding to obtained images are always available, unlike the potential unavailability of images obtained using the system 10 shown in FIG. 1 if the system 10 is busy being used for reviewing other images or examining other patients.

The distributed nature of the diagnostic imaging network 60 also allows the system to be quickly and inexpensively upgraded or modified because only the upgraded or modified component itself must be upgraded or modified. For example, if an improvement is made to a beamformer used in an ultrasound acquisition unit 90a, only the ultrasound acquisition unit 90a need be upgraded or replaced. Furthermore, the network 60 can be expanded simply by obtaining more of the component that is in need of expansion. For example, if there are enough display units 98 on the network 60 to view images in the desired locations, but not enough acquisition units 90 to obtain images, the system can be expanded simply by obtaining more acquisition units 90. Software upgrades or modifications can be made to the network 60 simply by upgrading or modifying the software residing on the system 62. Significantly, it is not necessary to upgrade or modify software residing in each of a larger number of systems as would be required with imaging systems of the type shown in FIGS. 1 and 2. Nor is it necessary to test or verify software installed in a large number of systems. If software resides in the acquisition units 90, the control units 94 or the display units 98, such software can be upgraded or modified simply by loading the software onto the system 62 and uploading the software from the system 62 to the other components on the network.

The distributed nature of the diagnostic imaging network 60 also allows the business of conducting examinations to be performed in a new and more advantageous manner. For example, since the system 62 is an integral part of each and every diagnostic examination, the hospital operating the diagnostic imaging network 60 can charge for the network 60 on a “per use” basis, such as a “per examination” or a “per image” or a “per unit of time” basis. Different charges can also be made for different uses of the system, such as a first charge for each image obtained using the system and a second charge for each viewing of an image using the network 60. The system 62 can be operated to keep track of each “per use” charge and, as previously mentioned, produce an invoice reflecting such charges. Charges by the manufacturer/distributor for the sale of the distributed system to the institution owning it can be based on time such as a monthly or annual fee, and/or can be based upon the number of clinical applications performed by the distributed system.

Charges for software upgrades can also made using a variety of techniques. The software upgrades can be paid for as part of the “per use” charges made for using the network 60. Alternatively, the software upgrade can be paid for with a single licensing fee or periodic licensing fees, or based upon the number and types of acquisition units 90 which may be connected to the network, and an upgrade can be provided for less than the entire network 60. For example, a display upgrade, which makes ultrasound images viewable with greater clarity, can be installed only on monitors that are used for viewing abdominal ultrasound images, where image clarity is very important, thus, in effect, charging a site license fee.

Distributed imaging systems present new approaches to conducting the business of selling, installing, and expanding the capabilities of an imaging site such as a clinic or hospital. In the past, a doctor needing diagnostic imaging system would order the system from a manufacturer or distributor and the ultrasound system would be shipped to the doctor's location, uncrated, and plugged into an a.c. outlet, ready for use. Other imaging systems, such as CT systems, X-ray, mammography and MRI systems and PET and nuclear cameras are sold and delivered in a similar manner, with the increased installation complexities of those systems. If a customer orders several diagnostic imaging systems, the multiple systems would be shipped and plugged in, in the same manner. To expand the imaging capabilities with another diagnostic imaging system, an additional diagnostic imaging system would be shipped and installed. If the clinic or hospital is networked so that patient information, setup protocols, images or reports can be communicated between systems, to workstations, and/or stored on a network storage device, the diagnostic imaging systems are connected to the network or modem connection at the time of installation.

But with distributed imaging systems, the sale and installation is approached much in the manner of that of a data network. The salesperson will counsel the customer as to the data handling requirements of the distributed imaging system and will explore whether the customer's existing network is sufficient to meet those needs. It would be desirable for the hospital or clinic to have an existing network with the speed, capacity, bandwidth, data processing, and interface capabilities suitable for the real time connection and data processing needs of the distributed imaging system, so that the customer can leverage his existing network and capabilities and reduce the cost of new data processors and networks. Desirably, the imaging software for the distributed system would run on an existing computer platform which would serve as the data processing system 62, and the display monitors already installed on the network could serve as the distributed system's display units 98. If the customer does not have the needed capability already in place, the salesperson may counsel the customer on a network expansion or new server that can be installed or added to the current hospital or clinic network to provide the needed capability. Once the network and computing hardware needed have been defined, the customer can order the types and numbers of acquisition units 90, control units 94, and/or display units 98 which provide the desired variety and number of virtual imaging systems and modalities which the distributed imaging system network will equivalently provide. If the customer later desires to expand those capabilities so that more or different imaging procedures can be done, the customer would simply order the additional acquisition units 90, control units 94, and/or display units 98 to provide the expanded or enhanced imaging capability. The image processing for the expanded capability would continue to be provided by the networked data processing system 62. If the customer desires to add a new functionality to the system which is performed or controlled by software, such as spatial compounding used in ultrasound imaging or resolution enhancement applicable to different modalities, for example, the software is installed on the data processing system 62, which effectively can upgrade every virtual imaging system of the network. Thus, multiple virtual imaging systems share a common networked processor or group of processors, and upgrades to that processor or group effectively upgrade every virtual system with a single software upgrade. The manufacturer or distributor no longer has to install upgrade software in each free-standing diagnostic imaging system in the hospital or clinic, which is the current practice, thereby providing greater efficiencies for both the serviceman and the hospital customer.

From the foregoing it will be appreciated that, although specific embodiments of the invention have been described herein for purposes of illustration, various modifications may be made without deviating from the spirit and scope of the invention. For example, while the embodiment of FIG. 3 indicates display units 98 at all patient locations on the network 60, it is understood that the display units, like the acquisition units 90 and the control units 94, can be mobile and can be stored at a central location or moved from one network connection to another as needed. The control unit and its controls can be integrated into either the display units or the acquisition units 90, or both. Thus, controls on the acquisition units and/or the display units can be used by the clinician during an examination to control imaging. For another example, as previously explained, although the imaging network 60 has been primarily described in the context of an ultrasound imaging system, it can also be implemented in the context of medical imaging systems of modalities other than ultrasound imaging systems, including x-ray systems, CT scan systems, digital radiography and mammography systems, PET and nuclear systems, MRI systems, etc. Accordingly, the invention is not limited except as by the appended claims.

1. A distributed medical imaging system comprising:
a plurality of acquisition probes structured to generate electrical image signals corresponding to a medical image;
a plurality of displays structured to display images responsive to electrical display signals;
a network having connections which interface with the acquisition probes and the displays; and
a data processor including data storage media coupled to the network to receive the electrical image signals from the acquisition probes and to apply the electrical display signals to the displays, the data processor interacting with connected ones of the acquisition probes to obtain image data corresponding to the electrical image signals, interacting with the data storage media to store the image data, and interacting with connected ones of the displays to cause the connected displays to display medical images corresponding to the stored image data.

2. The system of claim 1 wherein the data processor is structured to keep a record of the usage of the medical imaging system by keeping a record of the medical images obtained by the medical imaging system.

3. The system of claim 1 wherein the data processor is structured to keep a record of the usage of the medical imaging system by keeping a record of each patient examined from whom images are obtained using the medical imaging system.

4. The system of claim 1 wherein the data processor is further structured to prepare financial documents reflecting charges based on the record of the usage of the medical imaging system.

5. The system of claim 1 wherein at least a portion of the network comprises a hard-wired network.

6. The system of claim 1 wherein at least a portion of the network comprises a wireless network.

7. The system of claim 1 wherein the medical imaging system comprises an ultrasonic imaging system, and wherein the acquisition probes comprise ultrasonic imaging probes.

8. The system of claim 1 wherein the medical imaging system comprises a multimodality imaging system, and wherein the acquisition probes comprise acquisition devices of different diagnostic imaging modalities.

9. The system of claim 1, wherein the data processor comprises a plurality of data processing units.

10. The system of claim 1, wherein the data processor comprises a data processing unit which is a part of an integrated medical imaging system.

11. A distributed ultrasound system comprising:
an ultrasound signal acquisition device which connects to a network;
an acquisition device control in proximity to the ultrasound signal acquisition device;
an image display in proximity to the ultrasound signal acquisition device and connected to the network; and
a data processor physically remote from the acquisition device and coupled to the network which is responsive to input signals received over the network from the ultrasound signal acquisition device to perform signal and image processing and produces output image signals which are coupled over the network to the image display.

12. The distributed ultrasound system of claim 11 further comprising:
a second ultrasound signal acquisition device which connects to the network;
a second acquisition device control in proximity to the second ultrasound signal acquisition device; and
a second image display in proximity to the second ultrasound signal acquisition device and connected to the network;
wherein the data processor is responsive to input signals received over the network from the second ultrasound signal acquisition device to perform signal and image processing of the input signals received from the second ultrasound signal acquisition device in a time interleaved manner with the signals acquired from the first-named ultrasound signal acquisition device, and produces output image signals which are coupled over the network to the second image display.

13. The distributed ultrasound system of claim 12 wherein the data processor is responsive to input signals received over the network from the first-named and second ultrasound signal acquisition devices for the performance of the signal and image processing for a plurality of simultaneously conducted ultrasound examinations.

14. The system of claim 11, wherein the data processor comprises a plurality of data processing units.

15. The system of claim 11, wherein the data processor comprises a data processing unit which is a part of an integrated medical imaging system.

16. A method of conducting an ultrasound exam comprising:
locating an ultrasound signal acquisition device, an ultrasound system operating control, and a display device at a patient location;
connecting the ultrasound signal acquisition devices, the ultrasound system operating control, and the display device to an imaging device connection on a network which has a plurality of imaging device connections;
acquiring ultrasound signals with the acquisition device;
communicating the ultrasound signals over the network to a processor at a remote location;
processing the ultrasound signals with the processor to produce ultrasound image signals; and
communicating the ultrasound image signals over the network to the patient location for display on the display device.

17. The method of claim 16, wherein the ultrasound system operating control is integrated into one of the ultrasound signal acquisition device and the display device.

18. The method of claim 16, further comprising performing beamforming at the patient location prior to communicating the ultrasound signals over the network to the processor.

19. The method of claim 16, wherein processing further comprises performing signal and image processing with the processor.

20. The method of claim 16, further comprising communicating scanning control signals over the network from the processor to the ultrasound signal acquisition device.

21. A method of conducting diagnostic imaging examinations in a health care facility comprising:
locating a first diagnostic imaging signal acquisition device and a first display device at a first patient location;
locating a second diagnostic imaging signal acquisition device and a second display device at a second patient location;
connecting the acquisition devices and the display devices to imaging device connections on a network which has a plurality of imaging device connections;
acquiring diagnostic signals with the acquisition devices;
communicating the diagnostic signals over the network to a processor located in the health care facility;
processing the diagnostic signals from the acquisition devices with the processor to produce image signals; and
communicating the image signals over the network to the patient locations for display on the display devices.

22. The method of claim 21, wherein processing and communicating the signals of the respective acquisition devices and display devices is done in a time interleaved manner.

23. The method of claim 21, wherein communicating the image signals over the network further comprises communicating the image signals over the network to the patient locations for display on the display devices in substantially real time.

24. The method of claim 21, wherein acquiring further comprises acquiring diagnostic signals of a first imaging modality with the first signal acquisition device and acquiring diagnostic signals of a second imaging modality with the second signal acquisition device.

25. The method of claim 24, wherein processing further comprises performing image processing of diagnostic signals of a plurality of different imaging modalities with the processor.

26. The method of claim 21, further comprising installing new software on the processor, wherein the new software can be used in the conduct of diagnostic imaging examinations at a plurality of different patient locations.

27. The method of claim 21, wherein locating a second diagnostic imaging signal acquisition device and a second display device comprises locating a second diagnostic imaging signal acquisition device and a second display device in the residence of a patient.

28. The method of claim 27, wherein connecting the acquisition devices and the display devices further comprises connecting an acquisition device to a wireless connection to the network.
